(19) United States
(12) Patent Application Publication
(10) Pub. No.: US 2021/0085336 A1
(43) Pub. Date: Mar. 25, 2021

(54) Focal Pressure Applicator to Cease Bleeding

(71) Applicant: Rush University Medical Center, Chicago, IL (US)

(72) Inventors: Dana MOUSSALLI, Chicago, IL (US); Sushma BHARADWAJ, Chicago, IL (US)

(73) Assignee: Rush University Medical Center, Chicago, IL (US)

(21) Appl. No.: 17/029,113

(22) Filed: Sep. 23, 2020

Related U.S. Application Data
(60) Provisional application No. 62/904,223, filed on Sep. 23, 2019.

(51) Int. Cl. A61B 17/132; A61B 17/135

(52) U.S. Cl. CPC A61B 17/1325; A61B 2017/12004; A61B 17/135

(57) ABSTRACT

Disclosed herein is a device and method for reducing or stopping the flow of blood in a patient. The patient may be treated with an intravenous line, such as a central line. The device may have a plunger including a pressure pad that applies pressure to the bleeding vein to reduce or stop the flow of blood in an engaged position of the plunger.

### RELATED APPLICATIONS

This application claims the benefit of and priority to U.S. Provisional Application No. 62/904,223, filed Sep. 23, 2019, the entire contents of which are hereby incorporated herein by reference.

### FIELD

The present disclosure relates to an apparatus and method for reducing or stopping blood flow from a vein or an artery of a patient, particularly a patient being treated with an intravenous line, including patients having a central line.

### BACKGROUND

The statements in this section merely provide background information related to the present disclosure and may not constitute prior art.

Hospitalized patients may undergo invasive procedures as part of their medical care. In some cases, central venous catheter (CVC), also known as a central venous access device (CVAD), may be employed. A CVC is a catheter placed into a large vein. There are several different types of central venous catheters, each used for a different purpose. These catheters can be placed in veins in the neck (internal jugular vein), chest (subclavian vein), groin (femoral vein), or through axillary veins in the arms (also known as a peripherally inserted central catheter, or PICC line.) The CVC is used to administer medication or fluids, measurement of blood flow (hemodynamic monitoring, blood draws, and a variety of other reasons. Hemodialysis catheters, a type of central line, are used for hemodialysis and vasopressors. In the United States alone, more than 5 million CVCs are inserted each year, and by some estimates, up to 24% of hospitalized patients may be treated by insertion of a central line.

As with many invasive procedures, CVC use carries a risk of hemorrhage, and when patients undergo CVC insertion, there may be bleeding at the catheter exit point after the procedure is completed. Indeed, some patients may, due to their disease or condition, be at increased risk for bleeding from a CVC, but this is a risk even for otherwise healthy patients. The site can bleed for several hours, putting the patient at increased risk for infection such as central line associated blood infection (CLABSI), blood loss, anemia, and a host of other conditions.

Current practice recommendations for prevention and management of bleeding CVC lines vary depending on the institution. These practices include application of a sandbag, applying a pressure dressing, or using absorbable gelatin sponges (such as SURGIFOAM) to stanch bleeding. Additionally, in order to prevent bleeding after a procedure, physicians may attempt to correct coagulopathy prior to procedures with consequent use of expensive and/or limited resources, such as transfusion with fresh frozen plasma or platelets.

Each of these methods is accompanied by a number of risks and disadvantages. Administration of blood products carry risks, including transfusion related circulatory overload and acute hemolysis. Sandbags can cause discomfort to the patient because of their heavy weight, and can roll off the site and be displaced by patient movement. In some cases, use of a sandbag may be ineffective to stop bleeding independent of any additional risks. Absorbable gelatin sponges can lead to irritation due to frequent dressing changes, and this course of action generally requires coordination with a physician and pharmacy in order to dispatch the formulation. A pressure dressing does not allow for direct visualization of the puncture site, which may lead to delayed assessment for infection and bleeding, and further provides circumferential pressure, which may affect blood flow to the limbs, rather than merely addressing the bleeding at the CVC. Frequent dressing changes lead to excess hospital materials being used, and also puts patients at higher risk for infection. In all cases, nurses, physicians, or other hospital staff may need to remain with the patient until bleeding stops, which can take hours or even a full day. In turn, the remainder of the floor may become short-staffed as one or more of the hospital personnel remain holding a sandbag in place, or applying pressure at the site of bleeding and reapplying dressings.

It has been a challenge to develop a method of reducing or stopping blood flow from a newly placed CVAD.

### SUMMARY

According to one aspect of the present disclosure, a medical device for applying pressure to a body vessel in a patient is described. The medical device may include a base, which may include a first face and a second face opposite the first face. The base may define an aperture through the first face and the second face. The medical device may include a plunger disposed through the aperture, the plunger being movable from a retracted position to an engaged position. The plunger may include a pressure pad, which may be disposed on the second face of the base, such that when the plunger is moved from the retracted position to the engaged position, the pressure pad applies increased pressure to the body vessel. The plunger may also include a handle disposed on the first face of the base.

According to another aspect of the present disclosure, a medical device for applying pressure to a body vessel in a patient is described. The medical device may include a base which may include a first face and a second face opposite the first face. The base may define an aperture through the first face and the second face. The medical device may include a plunger disposed through the aperture. The plunger may be movable from a retracted position to an engaged position. The plunger may include a handle disposed on the first face of the base. The handle may be movable toward the retracted position when rotated in a first rotational direction and toward the engaged position when rotated in a second rotational direction. The plunger may include a pressure pad disposed on the second face of the base and including a rounded edge for contacting the patient. The plunger may include a spring connecting the handle to the pressure pad. The plunger may have a structure such that when the handle is moved vertically from the retracted position to the engaged position, the pressure pad applies increased pressure to the body vessel.

According to another aspect of the present disclosure, a method of applying pressure to a body vessel of a patient is described. The method may include a step of placing a medical device over the body vessel. The medical device may include a base having a first face and a second face opposite the first face. The base may define an aperture through the first face and the second face. The medical device may include a plunger disposed through the aperture, the plunger being movable from a retracted position to an engaged position. The plunger may include a pressure pad connected to the handle, disposed on the second face of the base, such that when the plunger is moved from the retracted position to the engaged position, the pressure pad applies increased pressure to the body vessel. The medical device may be placed in the retracted position, and placed such that the pressure pad is positioned over the body vessel. The method may further include manipulating the plunger to move the pressure pad to apply a focal pressure to the body vessel, such that the medical device is in the engaged position.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 is a perspective view of a medical device constructed in accordance with the principles of the present disclosure showing the first face of the base;

FIG. 2 is a perspective view of the medical device of FIG. 1, showing the second face of the base;

FIG. 3 is a top view of the medical device of FIG. 1;

FIG. 4 is a perspective view of a medical device constructed in accordance with the principles of the present disclosure, having a different form of a base;

FIG. 5 is a side view of a medical device of the present disclosure shown in the retracted position;

FIG. 6 is a side view of the medical device of FIG. 5, shown in the engaged position;

FIG. 7 is a cross-sectional view of a medical device constructed in accordance with the principles of the present disclosure;

FIGS. 8A and 8B are exploded views of another medical device constructed in accordance with the principles of the present disclosure;

FIG. 9 is a view of another medical device constructed in accordance with the principles of the present disclosure, having an inflatable element, the inflatable element illustrated in a deflated configuration;

FIG. 10 is a view of the device illustrated in FIG. 9, the inflatable element illustrated in an inflated configuration;

FIG. 11 is a top view of a base for use with a medical device; and

FIG. 12 is an illustration of method steps for stopping or reducing blood flow from a vein of a patient.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

Hereinafter, the present disclosure will be described in detail with reference to the accompanying drawings. In adding reference denotations to elements of each drawing, although the same elements are displayed on a different drawing, it should be noted that the same elements have the same denotations. In addition, in describing one aspect of the present disclosure, if it is determined that a detailed description of related well-known configurations or functions blurs the gist of one aspect of the present disclosure, it will be omitted.

In describing elements of the present disclosure, the terms 1^{st}, 2^{nd}, first, second, A, B, (a), (b), and the like may be used herein. These terms are only used to distinguish one element from another element, but do not limit the corresponding elements irrespective of the nature or order of the corresponding elements.

Unless otherwise defined, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

As used herein, the term “about,” when used in the context of a numerical value or range set forth means a variation of ±15%, or less, of the numerical value. For example, a value differing by ±15%, ±14%, ±10%, or ±5%, among others, would satisfy the definition of “about.”

None of the conventional solutions to bleeds at central lines mentioned above involve the application of focal pressure to the bleeding vessel. Focal pressure is pressure applied to a relatively small area, and allows for the specific application of the proper amount of pressure at the intended location.

In contrast, the pressure provided by a sandbag is non-focal, applying pressure across a large area that includes the bleeding vessel but also other portions of the body. The sandbag is inelegant, as it applies pressure non-selectively, and in many instances, will not be able to stop bleeding post CVC placement in the first place.

Likewise, the pressure provided by a pressure dressing is circumferential, rather than focal. This circumferential pressure achieves the intended function (stanching blood flow from the bleeding vein) but can also cut off blood flow to other, unintended vessels.

Veins are suitable target vessels for the application of focal pressure. Briefly, compared to arteries, veins are thin-walled and fibrous while having a larger diameter. This is due, in part to a much thinner tunica media layer in veins. In general, venous pressure is equivalent to the vascular pressure in a vein or in the atria of the heart, and is lower than arterial pressure, having common values of about 5 millimeters of mercury (mm Hg) in the right atrium and 8 mm Hg in the left atrium. The main determinants of venous blood pressure are the volume of fluid within the veins, and the compliance or distensibility of the vessel walls. Therefore, a vein is generally compressible because of the thinner tunica media and low blood pressure. As a result, veins, including a vein in which a central line has been placed, may be able to have bleeding stopped therefrom by the application of focal pressure, which will compress the vein, subsequently initiating the clotting cascade and stopping the bleeding.

Arteries are likewise suitable target vessels for the application of focal pressure. Arterial bleeds may be difficult to control in the absence of a significant amount of pressure to compress the vessel. However, in an artery and a vein of the same size, on balance more pressure will be applied to stop a bleed from the artery; for example, a pressure of up to about 75 mm Hg, or of about 80 mm Hg, or of about 90 mm Hg, or of about 100 mm Hg, or of about 110 mm Hg, or of about 120 mm Hg, or of about 130 mm Hg, or of about 140 mm Hg, or of about 150 mm Hg, or of about 160 mm Hg, or of about 170 mm Hg, or of about 180 mm Hg, may be applied to an artery in order to slow or stop arterial bleeding.

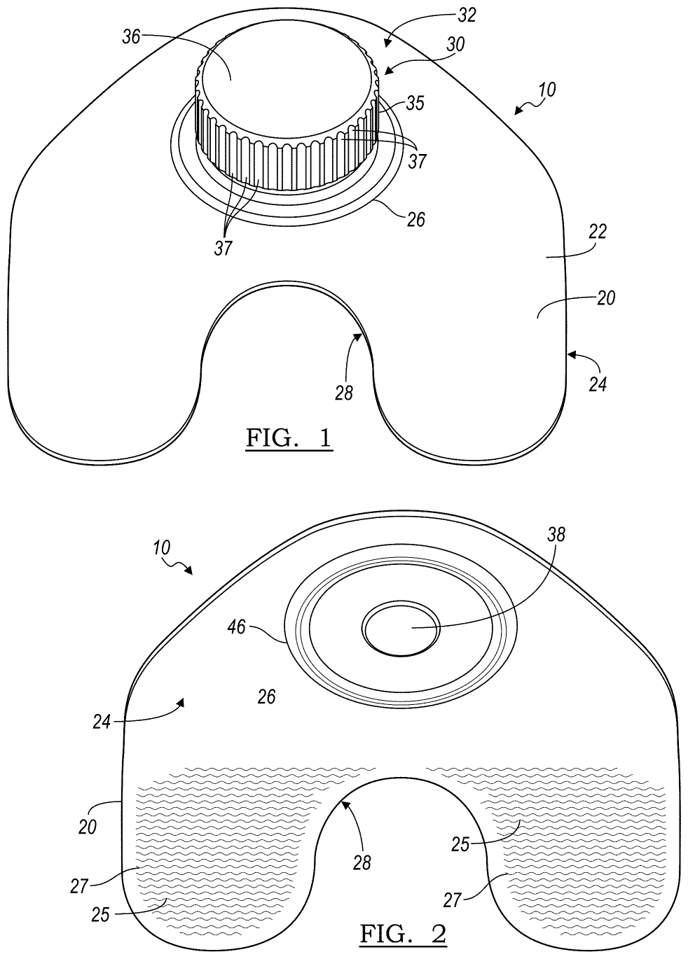

FIG. 1 illustrates one form of a medical device 10 which can apply focal pressure to a bleeding vein. The medical device 10 may include base 20, which has first face 22 and second face 24 opposite first face 22 (best seen in FIG. 2). The base may have an aperture 26 that extends through first face 22 and second face 24, defining a void in the base 20. The base 20 may be made of a soft or pliable material, such as a plastic or a polymer, for increased comfort for the patient. The second face 24 of base 20 may be capable of gently gripping the skin, or may have a “sticky” or textured surface 25, either on the whole surface or on a portion 27 thereof, so that the medical device 10 does not slip or move after placement by a medical practitioner.

As illustrated in FIGS. 1 and 2, the base 20 may have a shape that resembles a capital letter A, due to the optional indentation 28. Indentation 28 allows for the placement of medical device 10 over a target vessel while still permitting access to and visibility of the insertion site of the central line.

The medical device 10 may also include plunger 30. As seen in FIGS. 1 and 2, the plunger 30 may be placed through the aperture 26 and may extend from the side of the medical device 10 which includes the first face 22 to the side which includes second face 24. As described herein, a portion of the plunger 30 which extends through the aperture 26 such that it is on the side of base 20 which faces the first face 22 is said to be disposed on the first face 22, and a portion of the plunger 30 which faces the second face 24 is said to be disposed on the second face 24.

The plunger 30 may include a handle 36, which in one aspect is rotatable so as to move the plunger 30 and the medical device 10 from a retracted position 32 (shown in FIG. 5) to an engaged position 34 (shown in FIG. 6), and vice versa. The handle 36 may define a round shape, as shown in the figures, and may include a plurality of vertical grooves 37 about its circumference to provide a better or more comfortable grip for a medical practitioner. The handle 36, or a region around the handle 36, may be marked with a numbering and a pointer to indicate the amount of focal pressure that is delivered by the medical device 10 when the handle 36 is rotated to a given position.

As seen in FIG. 2, the plunger 30 may also include a pressure pad 38. The pressure pad 38 is another surface of medical device 10 that comes into contact with the body of the patient. The pressure pad 38 contacts the patient in the engaged position 34, and may also contact the patient in retracted position 32, albeit applying less pressure in this case.

The medical device 10 may include support ring 46, which may be formed about the aperture 26, or may be formed in the aperture 26 in direct contact with the base 20. The support ring 46 may assist in keeping the pressure pad 38 from contacting the body of the patient in the retracted position 32. The support ring 46 may be made of a plastic or a polymer, and may be pliable, providing a soft edge for patient comfort.

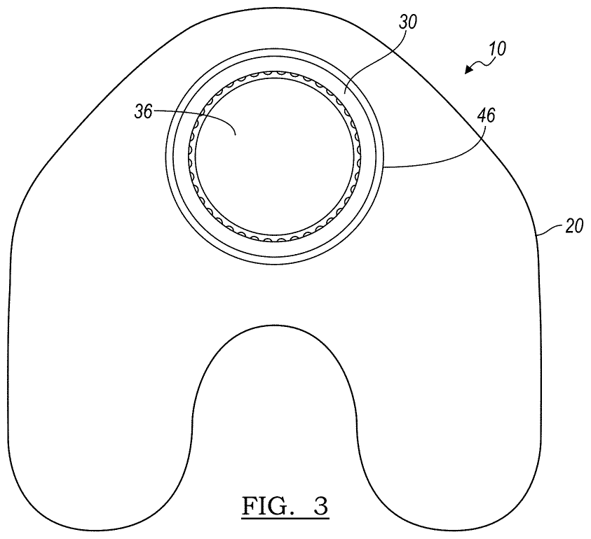

FIG. 3 provides a top view of a form of medical device 10, showing the support ring 46 bounding plunger 30.

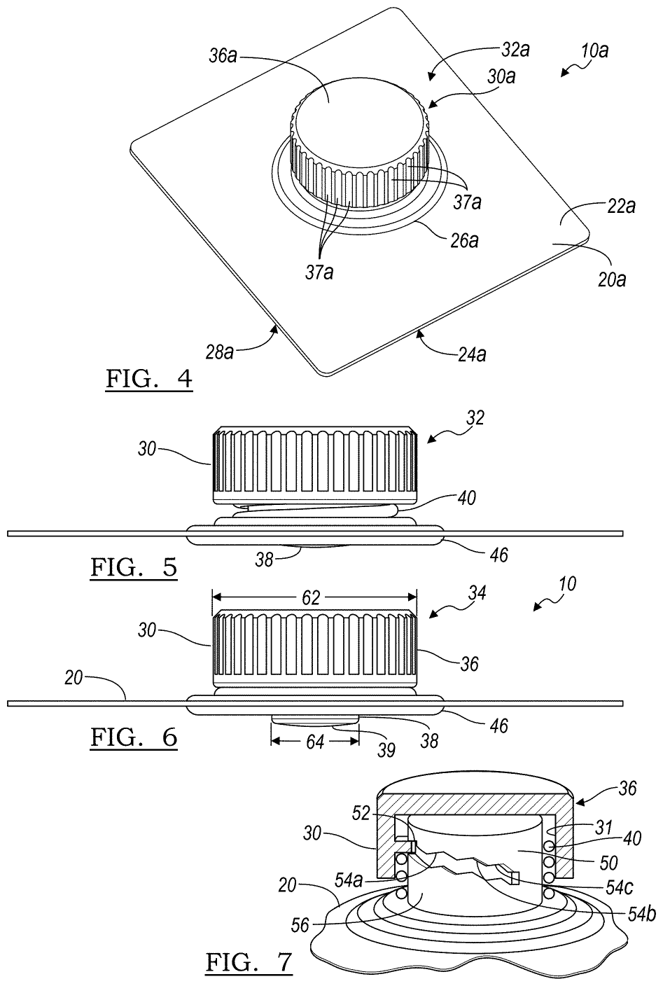

FIG. 4 illustrates another aspect of a medical device 10a constructed in accordance with the principles of the present disclosure. The principal difference between FIG. 4 and the device shown in FIGS. 1-3 is that base 20a is of a rectangular shape, rather than an A shape. The base 20a, as shown, lacks an indentation as in FIG. 1, but in some aspects may be formed with an indentation. The base 20a may be provided on the medical device 10 with any shape depending on the portion of the body it is intended to treat.

FIGS. 5-7 illustrate an aspect of the medical device 10, with particular focus on the plunger 30. The medical device 10 in this aspect features a spring-loaded plunger 30 for applying focal pressure. The spring 40 may be seen in FIG. 5. The plunger 30 and the medical device 10 are shown in FIG. 5 in retracted position 32. In the retracted position 32, only a small portion of pressure pad 38 can be seen extending beyond support ring 46.

At one end, the spring 40 is attached to handle 36, and at the other end, it is attached to pressure pad 38. The spring 40 is selected to have a spring constant capable of producing sufficient force to generate the desired pressure on a bleeding vessel. In one aspect, the pressure applied to a superficial, or near-surface, vessel may be between about 15 mm Hg and about 45 mm Hg, or between about 20 mm Hg and about 35 mm Hg, or between about 25 mm Hg and about 30 mm Hg. In general, veins have about 10-12 mm Hg vascular pressure. In the case of a deep vein, an applied focal pressure greater than this threshold may cause occlusion, for instance a pressure of between about 15 mm Hg and about 70 mm Hg, or between about 20 mm Hg and about 60 mm Hg, or between about 25 mm Hg and about 50 mm Hg, or between about 30 mm Hg and about 45 mm Hg, or about 35 mm Hg, or about 40 mm Hg, or any value between about 15 mm Hg and about 70 mm Hg.

FIG. 6 illustrates the device of FIG. 5 in engaged position 34. Rotating the handle 36 has resulted in vertical movement of the handle 36 toward the base 20 of the medical device 10, compressing spring 40 and causing the vertical extension of pressure pad 38 toward the patient. As can be seen in FIG. 6, pressure pad 38 may include a rounded edge 39 for contacting the patient. The pressure pad 38, in this aspect, has a smaller pad diameter 64 than the handle diameter 62. The small surface area of the pressure pad 38 allows for the application of ample focal pressure on a predetermined portion of the patient's anatomy. The pad diameter 64 may be varied depending on the treatment site.

The medical device 10 and the plunger 30 can be moved from the retracted position 32 into the engaged position 34 by rotation of the handle 36 in a first rotational direction, and from the engaged position 34 to the retracted position 32 by rotation of the handle 36 in a second rotational direction which is opposite the first rotational direction.

The plunger 30 of medical device 10 of FIG. 5 is illustrated in cross section in FIG. 7. In this aspect, the handle 36 defines an empty interior space in which a support cylinder 50 may be disposed. The spring 40 surrounds the support cylinder 50. The top of support cylinder 50 defines a stopping distance for the maximum vertical descent of the handle 36.

The support cylinder 50 may have an outer surface 56, in which a channel 52 may be formed. As illustrated, the channel 52 features a plurality of detents 54a/54b/54c, but in other aspects, the channel 52 may instead be smooth or continuous along its length. In turn, protrusion 60 is provided on an inner surface 31 of the handle 31. The protrusion 60 is sized to fit in channel 52 and slide along its length as the handle 36 is turned. The detents 54a/54b/54c represent positions into which the handle can be “locked,” allowing the medical practitioner to easily supply focal pressures of several predetermined magnitudes to the vessel based on need. For example, the spring constant and the vertical height of each detent may be designed such that locking the handle 36 into detent 54a supplies a focal pressure of about 25 mm Hg to the vessel to be treated, locking into detent 54b supplies a focal pressure of about 30 mm Hg, and locking into detent 54c supplies a focal pressure of about 35 mm Hg.

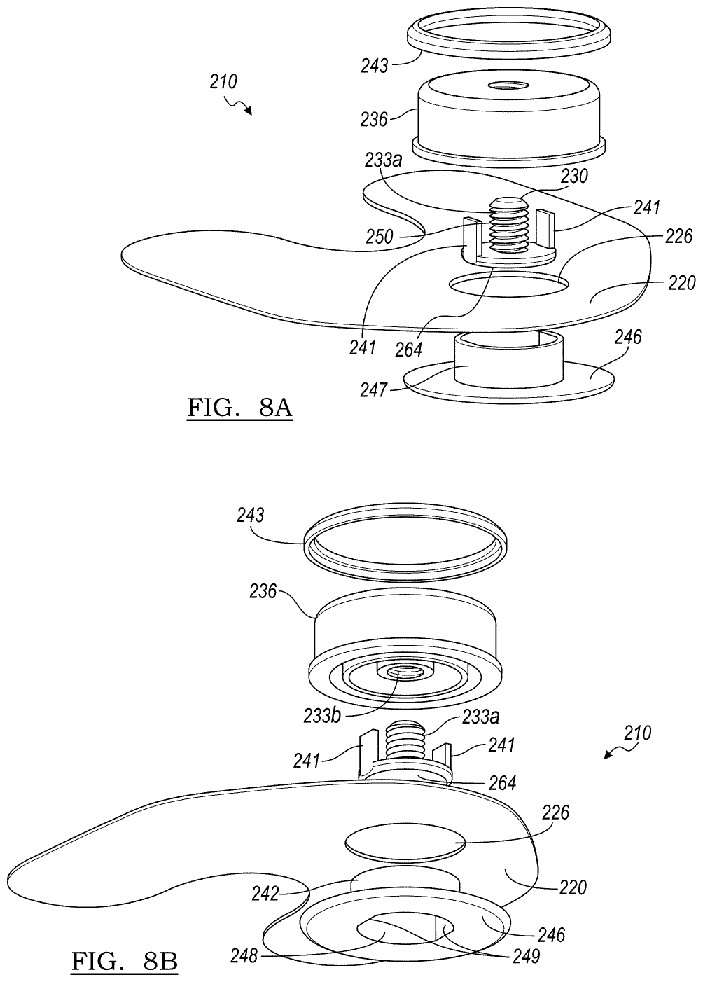

A medical device 210 constructed in accordance with the principles of the present disclosure is illustrated in FIGS. 8A and 8B. In this aspect, the support cylinder 250 is a portion of the plunger 230, rather than of a support ring, and the pressure pad 264 is formed on a bottom surface of the support cylinder 250. On its outer surface, the support cylinder 250 of the plunger 230 may have one or more first threads 233a formed on its outer surface, rather than a channel. The first threads 233a can extend inward toward the center of the support cylinder (that is, they are depressions or indentations), or can extend outward from the outer surface of the support cylinder 250. These threads can engage corresponding second threads 233b on the inside of the handle 230 in threaded fashion. The plunger 230 may operate by a spring positioned, in one aspect, about the support cylinder 250, as in the device of FIGS. 4-7. The control of the device by rotation of the handle may be as described previously.

The plunger 236 may include at least one tab 241 set off-center from the support cylinder 250. In FIG. 8A, there are two tabs 241, but there may be only one, or there may be more than two. These tabs 241 can engage flats 249 of the cylindrical portion 247 of support ring 246, which extends through the aperture 226 of base 220, and has a cylinder lumen 248 which allows passage of the plunger 230 therethrough. The flats 249 hold the tabs 241 in place during rotation of the handle 236, inhibiting rotational movement of the plunger 230. Securement ring 243 assists in holding the plunger assembly together with the base 220.

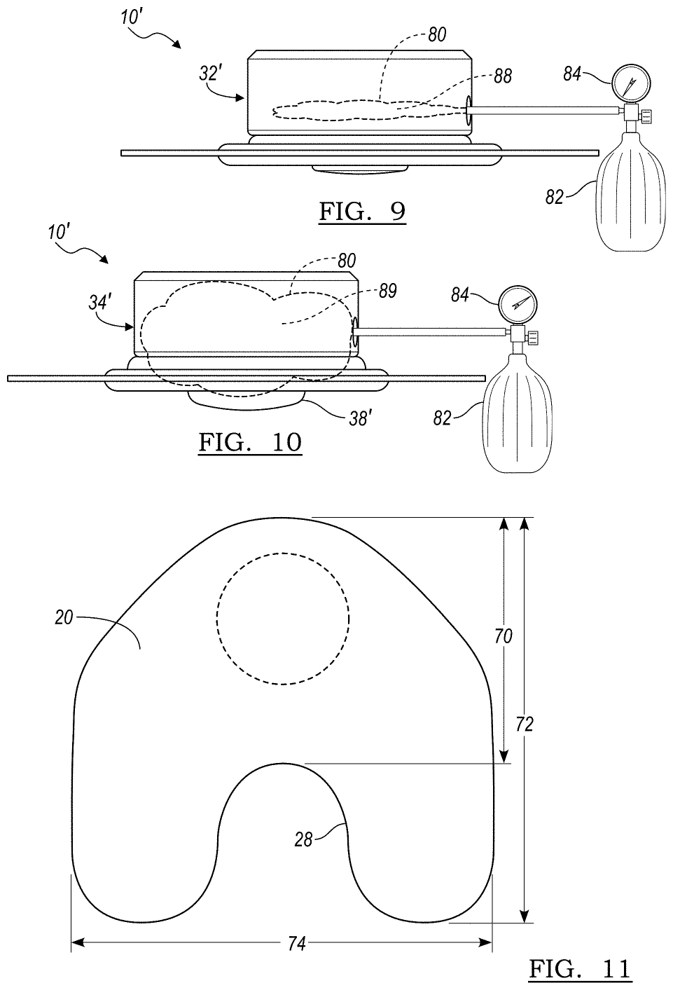

A medical device according to the present disclosure may produce focal pressure using means other than a spring. For example, a medical device 10′ as shown in FIGS. 9 and 10 may use an inflatable element 80, such as a balloon, to provide focal pressure. The medical device 10′ is shown in retracted position 32′ in FIG. 9, with the inflatable element 80 in a deflated state 88. The inflatable element 80 is in fluid connection with a fluid source, such as bulb 82 (akin to the bulb of a sphygmomanometer), or a gas source and may include a pressure gauge 84. When the inflatable element 80 is inflated to inflated state 89, as shown in FIG. 10, the inflatable element 80 contacts the pressure pad 38′ and forces the pressure pad 38′ vertically toward the patient. When focal pressure is no longer needed, the fluid or gas can be released, deflating inflatable element 80. The pressure gauge 84 may be adjusted to show the focal pressure being applied to the vessel rather than the internal pressure of the inflatable element 80.

In another aspect, the pressure pad 38′ may be a component of inflatable element 80, such that the two components are physically joined.

The medical device 10′ of FIGS. 9-10 need not have a rotatable handle, as the focal pressure is not generated by rotation, but by filling the inflatable element 80 with gas or liquid.

It will be noted that a device in accordance with the principles of the present disclosure may be constructed using features from any combination of the various handles and plungers illustrated in FIGS. 4-10.

FIG. 11 illustrates the dimensions of the base 20 in one aspect of the present device. In one example, length 72 can be about 10.7 centimeters (cm), and width 74 can be about 9.0 cm, making the footprint of the device relatively compact: less than 100 square cm, including voids. The distance 70 between a peak of the indentation 28 and a peak of the base 20 can be about 7.4 cm. All dimensions can be varied as best suits the particular application.

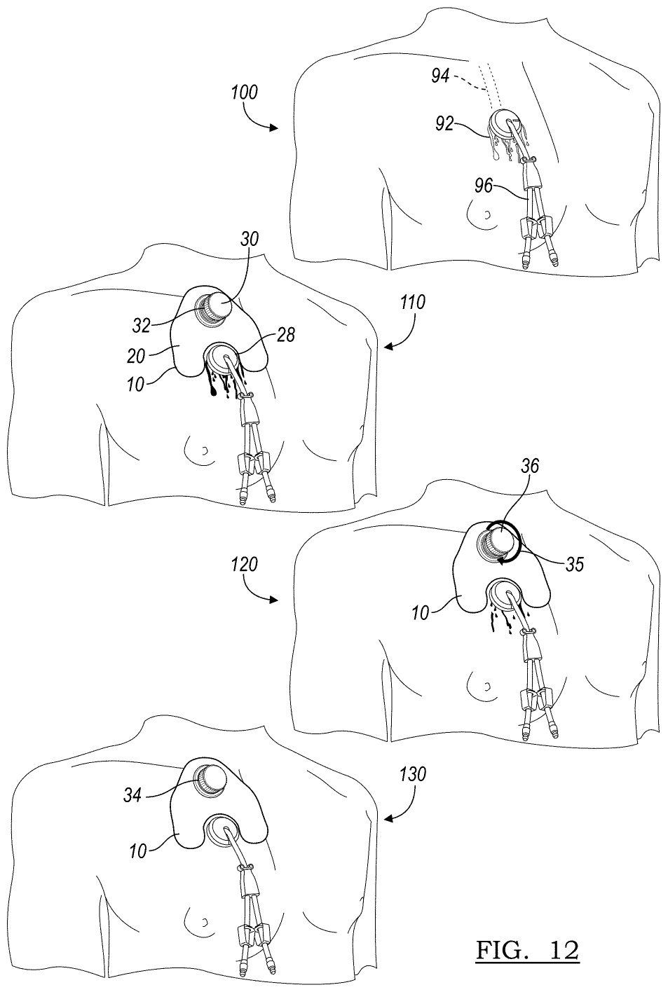

A method for treatment of a bleeding vein having an intravenous line is shown in FIG. 12. In step 100, the bleeding 92 is recognized by a medical practitioner in a patient having central line 96 inserted into vein 94. In step 110, a medical device 10 having plunger 30 in base 20, as described herein, is placed with pressure pad 38 positioned over the vein 94. The medical device 10 is in retracted position 32. The insertion site of central line 96 is visible due to placement of the central line 96 within indentation 28 of base 20.

In step 120, the handle 36 is rotated 35 in a first rotational direction. Finally, in step 130, when the handle 36 has been rotated sufficiently, the medical device 10 is in engaged position 34, providing focal pressure to vein 94. The focal pressure may be from about 15 mm Hg to about 45 mm Hg, or from about 20 mm Hg to about 35 mm Hg, or from about 25 mm Hg to about 30 mm Hg. When the appropriate pressure is reached, bleeding is reduced or ceases.

A medical device as described herein which can apply focal pressure to a bleeding vein, thereby stopping bleeding, can allow for control of bleeding before or after a CVC dressing is applied. Bleeding at the CVC site is associated with increased risk of infection, and such a medical device avoids the accumulation of fluid-soaked dressing at the site. Quick and effective cessation of bleeding inhibits blood volume loss, reduces dressing changes, and represents an improvement in patient care, freeing up medical practitioners who would otherwise need to remain holding a sandbag in place or applying dressing or foam. In turn, this may result in a financial benefit to the hospital, as staff will be relieved of additional duties due to the device, and fewer physical resources (CVC dressing kits, stat locks for PICCs, and so forth) will be consumed by bleeds at central lines.

Although the present disclosure has been described with reference to examples and the accompanying drawings, the present disclosure is not limited thereto, but may be variously modified and altered by those skilled in the art to which the present disclosure pertains without departing from the spirit and scope of the present disclosure.

What is claimed is:

1. A medical device for applying pressure to a body vessel in a patient, the medical device comprising:
a base comprising a first face and a second face opposite the first face, the base defining an aperture through the first face and the second face; and
a plunger disposed through the aperture, the plunger being movable from a retracted position to an engaged position, the plunger comprising a pressure pad disposed on the second face of the base, such that when the plunger is moved from the retracted position to the engaged position, the pressure pad applies increased pressure to the body vessel.

2. The medical device of claim 1, wherein the plunger further comprises a handle disposed on the first face of the base.

3. The medical device of claim 2, the handle is operably connected to the pressure pad by a spring.

4. The medical device of claim 2, wherein the handle moves toward the retracted position when rotated in a first rotational direction, and wherein the handle moves toward the engaged position when the handle is rotated in a second rotational direction.

5. The medical device of claim 2, wherein the handle defines a round shape and comprises a plurality of grooves formed along a circumference thereof.

6. The medical device of claim 1, wherein the pressure pad comprises a rounded edge for contacting the patient.

7. The medical device of claim 1, further comprising a support ring disposed in the aperture of the base.

8. The medical device of claim 7, further comprising a support cylinder extending from the support ring and disposed within a handle of the plunger, the support cylinder having an outer surface defining a channel therein.

9. The medical device of claim 8, wherein the handle comprises an inner surface, the inner surface comprising a protrusion formed thereon, the protrusion being sized to engage the channel.

10. The medical device of claim 8, wherein the channel defines a plurality of detents, the handle and pressure pad being held at a position intermediate of the engaged position and the retracted position when the protrusion is engaged with one of the plurality of detents.

11. The medical device of claim 7, wherein the plunger comprises a support cylinder extending into the handle, the support cylinder having an outer surface on which at least one first thread is formed, the handle comprising an inner surface on which at least one second thread is formed, the at least one second thread being configured to engage with the at least one first thread.

12. The medical device of claim 1, wherein the pressure pad defines a first diameter, and the handle defines a second diameter greater than the first diameter.

13. The medical device of claim 1, wherein the base defines an indentation sized to accommodate an intravenous line.

14. The medical device of claim 12, wherein the intravenous line is a central line.

15. The medical device of claim 1, wherein the pressure pad applies a focal pressure of about 15 mm Hg to about 180 mm Hg to the body vessel when the medical device is in the engaged position.

16. The medical device of claim 1, wherein the pressure pad applies a focal pressure of about 15 mm Hg to about 70 mm Hg to the body vessel when the device is in the engaged position.

17. The medical device of claim 1, wherein the base comprises a polymer material.

18. The medical device of claim 1, wherein the plunger further comprises an inflatable element, the inflatable element operably connected to the pressure pad.

19. A medical device for applying pressure to a body vessel in a patient, the medical device comprising:
a base comprising a first face and a second face opposite the first face, the base defining an aperture through the first face and the second face; and
a plunger disposed through the aperture, the plunger being movable from a retracted position to an engaged position, the plunger comprising:
a handle disposed on the first face of the base, the handle being adapted to move the plunger toward the retracted position when rotated in a first rotational direction and toward the engaged position when rotated in a second rotational direction,
a pressure pad, the pressure pad being disposed on the second face of the base and comprising a rounded edge for contacting the patient, and
a spring operably connecting the handle to the pressure pad,
such that when the handle moves the plunger from the retracted position to the engaged position, the pressure pad applies increased pressure to the body vessel.

20. A method of applying pressure to a body vessel of a patient, the method comprising:
placing a medical device over the body vessel, the medical device comprising:
a base comprising a first face and a second face opposite the first face, the base defining an aperture through the first face and the second face; and
a plunger disposed through the aperture, the plunger being movable from a retracted position to an engaged position, the plunger comprising a pressure pad disposed on the second face of the base, such that when the plunger is moved from the retracted position to the engaged position, the pressure pad applies increased pressure to the body vessel;
the medical device being in the retracted position and placed such that the pressure pad is positioned over the body vessel; and
manipulating the plunger to move the pressure pad to apply a focal pressure to the body vessel, such that the medical device is in the engaged position.

21. The method of claim 20, wherein the plunger further comprises a handle disposed on the first face of the base.
